(19) United States
(12) Patent Application Publication
(10) Pub. No.: US 2005/0171161 A1
(43) Pub. Date: Aug. 4, 2005

(54) Method of treatment or prevention of obesity

(76) Inventors: Tung M Fong, RAHWAY, NJ (US); Leonardus H. T. Van Der Ploeg, Scotch Plains, NJ (US)

Correspondence Address: MERCK AND CO., INC, P O BOX 2000, RAHWAY, NJ, 07065-0907, US

(21) Appl. No.: 10/506,395

(22) PCT Filed: Feb. 28, 2003
(86) PCT No.: PCT/US03/06031

Related U.S. Application Data
(60) Provisional application No. 60/362,275, filed on Mar. 6, 2002.

(51) Int. Cl. A61K 31/454

(52) U.S. Cl. 514/326

(57) ABSTRACT

The present invention relates to a method of treating or preventing obesity (or suppressing the appetite) in a human patient by antagonizing CB1 receptors and inhibiting the enzyme 11β-HSD1 in an amount that is effective to treat or prevent obesity. Compounds useful in the present invention have an ion channel activity level greater than about 2 μM. Preferably the compound is a dual selective inhibitor, selectively antagonizing CB1 receptors and selectively inhibiting the enzyme 11β-HSD1

### BACKGROUND OF THE INVENTION

The present invention relates to a method of treating or preventing obesity and related feeding and other metabolic disorders in a human patient by antagonizing CB1 receptors and inhibiting the enzyme 11β-HSD1 in an amount that is effective to treat or prevent obesity or such other feeding and metabolic disorders.

Antagonists of the Cannabinoid-1 (CB1) receptor and inhibitors of the enzyme 11β-hydroxy steroid dehydrogenase-1 (11β-HSD type 1) as described herein are useful for the treatment or prevention of obesity and related metabolic disorders. Moreover, the methods described herein provide for a surprisingly low level of activity across ion channels, which is useful in reducing side effects.

Marijuana (Cannabis sativa L.) and its derivatives have been used for centuries. A major active ingredient in marajuana is Δ⁹-tetrahydrocannabinol (Δ⁹-THC). Detailed research has revealed that the biological action of Δ⁹-THC and other members of the cannabinoid family occurs through two G-protein coupled receptors termed CB1 and CB2. The CB1 receptor is primarily found in the central and peripheral nervous systems and to a lesser extent in several peripheral organs. The CB2 receptor is found primarily in lymphoid tissues and cells. Three endogenous ligands for the cannabinoid receptors derived from arachidonic acid have been identified (anandamide, 2-arachidonoyl glycerol, and 2-arachidonyl glycerol ether). Each is an agonist with activities similar to Δ⁹-THC, including sedation, hypothermia, intestinal immobility, antinociception, analgesia, catalepsy, anti-emesis and appetite stimulation.

Genes for the respective cannabinoid receptors have each been disrupted in mice. The CB1 receptor knockout mice appeared normal and fertile. They were resistant to the effects of Δ⁹-THC and demonstrated a strong reduction in the reinforcing properties of morphine and the severity of withdrawal syndrome. The mice also demonstrated reduced motor activity and hypoalgesia. The CB2 receptor knockout mice were also healthy and fertile. They were not resistant to the central nervous system mediated effects of administered Δ⁹-THC. There were some effects on immune cell activation, reinforcing the role for the CB2 receptor in immune system functions.

Specific synthetic ligands for the cannabinoid receptors have been developed and have aided in the characterization of the CB receptors: CP55,940 (J. Pharmacol. Exp. Ther. 1988, 247, 1046-1051); WIN55212-2 (J. Pharmacol. Exp. Ther. 1993, 264, 1352-1363); SR141716A (FEBS Lett. 1994, 350, 240-244; Life Sci. 1995, 56, 1941-1947); and SR144528 (J. Pharmacol. Exp. Ther. 1999, 288, 582-589). The pharmacology and therapeutic potential for cannabinoid receptor ligands has been reviewed (Exp. Opin. Ther. Patents 1998, 8, 301-313; Ann. Rep. Med. Chem., A. Doherty, Ed.; Academic Press, NY 1999, Vol. 34, 199-208; Exp. Opin. Ther. Patents 2000, 10, 1529-1538; Trends in Pharma. Sci. 2000, 21, 218-224). There is at least one CB1 receptor modulator characterized as an inverse agonist or an antagonist, N-(1-piperidinyl)-5-(4-chlorophenyl)-1-(2,4-dichlorophenyl)-4-methylpyrazole-3-carboxamide (SR141716A), in clinical trials for treatment of eating disorders at this time. There are two other compounds designated AM-251 and AM-281 which are CB1 receptor antagonists.

US patents U.S. Pat. No. 5,624,941 and U.S. Pat. No. 6,028,084, PCT Application Nos. WO98/43636 and WO98/43635, and EPO Application No. EP-658546 disclose substituted pyrazoles having activity against the cannabinoid receptors.

PCT Application Nos. WO98/31227 and WO98/41519 also disclose substituted pyrazoles having activity against the cannabinoid receptors.

PCT Application Nos. WO98/37061, WO00/10967, and WO00/10968 disclose diaryl ether sulfonamides having activity against the cannabinoid receptors.

PCT Application Nos. WO97/29079 and WO99/02499 disclose alkoxy-isoindolones and alkoxy-quinolones as having activity against the cannabinoid receptors.

US patent U.S. Pat. No. 5,532,237 discloses N-benzoyl-indole derivatives having activity against the cannabinoid receptors.

US patents U.S. Pat. No. 4,973,587, U.S. Pat. No. 5,013,837, U.S. Pat. No. 5,081,122, and U.S. Pat. No. 5,112,820, U.S. Pat. No. 5,292,736 disclose aminoalkylindole derivatives as having activity against the cannabinoid receptors.

The present invention further includes the use of compounds that are inhibitors of the enzyme 11β-HSD1. Generally, glucocorticoid concentrations are modulated by tissue-specific 11β-hydroxysteroid dehydrogenase enzymes. The two enzymes (also referred to as isozymes) of 11β-HSD (11β-HSD1 and 11β-HSD2) have different cofactor requirements and substrate affinities. Each has been successfully cloned in rat and human tissues. The 11β-hydroxysteroid dehydrogenase type 1 enzyme (11β-HSD1) is a low affinity enzyme that generally uses NADP+ as a cofactor rather than NAD+ (Agarwal et al., 1989). In vitro studies have shown that 11β-HSD1 is capable of acting as both a reductase and a dehydrogenase. However, 11β-HSD1 in vivo generally acts as a reductase, converting 11-ketoglucocorticoids, such as cortisone, to 11β-hydroxyglucocorticoids such as cortisol.

Excessive levels of cortisol have been associated with obesity, perhaps due to increased hepatic gluconeogenesis. Thus, the administration of an effective amount of an 11β-HSD1 inhibitor may be useful in the treatment or control of obesity. Long-term treatment with an 11β-HSD1 inhibitor may also be useful in delaying the onset of obesity, or perhaps preventing it entirely, especially if the patient uses an 11β-HSD1 inhibitor in combination with controlled diet and exercise.

There is a long standing need for the treatment or prevention of obesity, without causing side effects, such as CNS side effects, cardiovascular effects, such as hypertension, and the like. Using the combination of CB1 receptor antagonism and the inhibition of the enzyme 11β-HSD1, a method of treating or preventing obesity is attained.

### SUMMARY OF THE INVENTION

The present invention addresses a method of treating or preventing obesity in a human patient in need of such treatment or prevention, by antagonizing CB1 receptors and inhibiting the enzyme 11β-HSD1 in an amount that is effective to treat or prevent obesity.

More particularly, a method of treating or preventing obesity or related metabolic disorders in a human patient in need of such treatment or prevention is disclosed that is comprised of administering to said patient a compound that antagonizes the CB1 receptor and inhibits the enzyme 11β-HSD1 in an amount that is effective to treat or prevent obesity or the related metabolic disorder, said compound having an ion channel selectivity with activity levels greater than about 2 μM.

Even more particularly, a method of treating or preventing obesity or related metabolic disorders in a human patient in need of such treatment or prevention is disclosed which comprises administering to said patient a first compound that selectively antagonizes the CB1 receptor in combination with a second compound that selectively inhibits the enzyme 11β-HSD1, said compounds being administered in combination in an amount that is effective to treat or prevent obesity or the related metabolic disorder, said compounds having an ion channel activity level that is greater than about 2 μM.

The present invention is also concerned with the use of a compound or compounds for the manufacture of a medicament that is useful in treating or preventing obesity or to effect appetite suppression.

The present invention is also concerned with treatment or prevention of these conditions through a combination of compounds described herein, along with other currently available pharmaceuticals.

The invention is also concerned with pharmaceutical compositions comprising one or more of the compounds described herein in combination with a pharmaceutically acceptable carrier.

### DETAILED DESCRIPTION OF THE INVENTION

As used herein “obesity” refers to a condition whereby the patient has a Body Mass Index (BMI), which is calculated as weight per height squared (kg/m²), of at least 30. For humans, conventionally, those persons with normal healthy weight have a BMI of 19.9 to less than 25. Overweight persons have a BMI of 25 to less than 30. “A person at risk of obesity” is an overweight person with a BMI of 25 to less than 30.

Obesity as used herein may be due to any cause, whether genetic or environmental. Examples of related metabolic disorders that may accompany, result in, or cause obesity include overeating and bulimia, polycystic ovarian disease, craniopharyngioma, the Prader-Willi Syndrome, Frohlich's syndrome, Type II diabetes, GH-deficient subjects, normal variant short stature, Turner's syndrome, and other pathological conditions showing reduced metabolic activity or a decrease in resting energy expenditure as a percentage of total fat-free mass, e.g, children with acute lymphoblastic leukemia.

“Treatment” (of obesity) as used herein refers to administering a compound or compounds that antagonize or selectively antagonize CB1 receptors, and inhibit or selectively inhibit the enzyme 11β-HSD1, with a goal of reducing the BMI of the patient, preferably to less than about 30, and maintaining that weight for at least 6 months. The treatment may result in a reduction in food or calorie intake by the mammal, or the result may be from an induction of effective changes in energy expenditure and lipid partitioning.

“Prevention” refers to preventing obesity or the related maetabolic disorder from occurring if the treatment is administered prior to the onset of the condition in overweight persons with a BMI between 25 and 30. Moreover, if treatment is commenced in already obese subjects, such treatment is expected to prevent, or to prevent the progression of, the related metabolic disorder and the medical sequelae of obesity, such as, e.g., arteriosclerosis, Type II diabetes, polycystic ovarian disease, cardiovascular diseases, osteoarthritis, dermatological disorders, hypertension, insulin resistance, hypercholesterolemia, hypertriglyceridemia and cholelithiasis.

The invention described herein thus broadly entails the antagonism of CB1 receptors and inhibition of the 11β-HSD1 enzyme, in an amount that is effective to treat or prevent obesity.

In one aspect of the invention, this is achieved using a “dual inhibitor” i.e., a compound that antagonizes the CB1 receptor and also inhibits the enzyme 11β-HSD1.

More particularly, in a preferred embodiment of the invention, the dual inhibitor does not substantially modulate ion channel activity below an effective concentration of about 2 μM. Representative examples of ion channels that are substantially unaffected below about 2 μM include the Na, K and Ca ion channels.

In a further aspect, the dual inhibitor is a selective CB1 receptor antagonist, a selective inhibitor of enzyme 11β-HSD1, or preferably is selective for both the CB1 receptor and the 11β-HSD1 enzyme.

“Selective” as used herein refers to antagonist activity at the comparable cannabinoid receptor CB2 and inhibitory activity at the comparable dehydrogenase enzyme 11β-HSD2. Thus, when a compound is CB1 receptor selective, this means that it has little to no activity, i.e., it would require a high effective concentration, to antagonize the CB2 receptor. Similarly, when a compound is 11β-HSD1 selective, this means that it has little to no activity against the comparable enzyme, 11β-HSD2, such that it would require a high effective concentration to inhibit the enzyme 11β-HSD2. Preferably, the compounds used in the present invention are dual selective inhibitors, i.e., both CB1 receptor selective and 11β-HSD1 selective.

A further aspect of this portion of the invention is that the dual selective inhibitor has an ion channel activity concentration that is at least about 6 μM or higher. Thus the preferred dual inhibitor provides weak to very weak modulation of ion channels when used in vivo.

In another aspect of the invention, the antagonism of CB1 receptors and inhibition of the 11β-HSD1 enzyme are achieved using two active compounds, a first compound that is CB1 selective and a second compound that is 11β-HSD1 selective. Preferably with respect to this aspect of the invention, the compounds have little to no activity modulating ion channels, such that the compounds useful herein have an effective concentration in modulating ion channels at greater than about 2 μM.

More particularly, the method of treating or preventing obesity in a human patient in need of such treatment or prevention is comprised of administering a compound that does not substantially modulate ion channels selected from the Na, K and Ca ion channels. In this aspect of the invention, the compound preferably has an ion channel activity level in at least two of the three channels mentioned of about 4 μM or higher. More preferably, the compound has an ion channel activity level of about 6 μM or higher in all three ion channel classes. Thus, in this aspect of the invention, the compound has an ion channel activity level greater than about 6 μM in the Na, K and Ca ion channels.

In another aspect of the invention, a method of treating or preventing obesity in a human patient in need of such treatment or prevention wherein the compound is a selective antagonist of CB1 receptors and a selective inhibitor of the enzyme 11β-HSD1.

In another aspect of the invention, a method of treating or preventing obesity in a human patient is disclosed wherein the compound that antagonizes the CB1 receptor is at least about 10 fold selective for the CB1 receptor over the CB2 receptor. For a compound to be deemed “10 fold selective”, the IC50 for antagonizing CB1 receptors should be about 10 times lower than the IC50 for antagonizing CB2 receptors. For a compound to be “at least 10 fold selective”, the inhibitory concentration (IC50) for antagonizing CB1 receptors should be at least about 10 times lower than the inhibitory concentration (IC50) for antagonizing CB2 receptors.

In another aspect of the invention, a method of treating or preventing obesity in a human patient is disclosed wherein the compound that antagonizes the CB1 receptor is from about 10 fold selective to about 1000 fold selective for the CB1 receptor over the CB2 receptor.

In another aspect of the invention, a method of treating or preventing obesity in a human patient wherein the compound that selectively inhibits the enzyme 11β-HSD1 is at least about 10 fold selective for 11β-HSD1 over the enzyme 11β-HSD2.

In another aspect of the invention, a method of treating or preventing obesity in a human patient is disclosed wherein the compound that inhibits the enzyme 11β-HSD1 is from about 10 fold selective to about 1000 fold selective for the enzyme 11β-HSD1 over the enzyme 11β-HSD2.

In another aspect of the invention, a method of treating or preventing obesity in a human patient in need of such treatment or prevention is disclosed wherein the compound administered antagonizes the CB1 receptor at an IC50 of about 100 nM or less.

In another aspect of the invention, a method of treating or preventing obesity in a human patient in need of such treatment or prevention is disclosed wherein the compound administered selectively inhibits the enzyme 11β-HSD1 at an IC50 of about 100 nM or less.

In another aspect of the invention, a method of treating or preventing obesity in a human patient in need of such treatment or prevention is disclosed wherein the compound administered that antagonizes the CB1 receptor and inhibits the enzyme 11β-HSD1 does not substantially antagonize the receptor CB2.

In another aspect of the invention, a method of treating or preventing obesity in a human patient in need of such treatment or prevention is disclosed wherein the compound administered does not substantially inhibit the enzyme 11β-HSD2.

In another aspect of the invention, a method of treating or preventing obesity in a human patient in need of such treatment or prevention is disclosed wherein the compound administered has an IC50 against CB2 receptors of about 300 nM or higher.

In another aspect of the invention, a method of treating or preventing obesity in a human patient in need of such treatment or prevention is disclosed wherein the compound administered has an IC50 against the enzyme 11β-HSD2 of at least about 1 μM.

In another aspect of the invention, a method of treating or preventing obesity in a human patient in need of such treatment or prevention is disclosed which is comprised of administering to said patient a first compound that selectively antagonizes the CB1 receptor in combination with a second compound that selectively inhibits the enzyme 11β-HSD1, said compounds being administered in combination in an amount that is effective to treat or prevent obesity, said compounds having an ion channel activity level that is greater than about 2 μM.

In another aspect of the invention, a method of treating or preventing obesity in a human patient in need of such treatment is disclosed wherein said compounds have an ion channel activity level that is greater than about 2 μM.

In another aspect of the invention, a method of treating or preventing obesity in a human patient in need of such treatment or prevention, is disclosed wherein the ion channel is a Na, K or Ca ion channel.

In another aspect of the invention, a method of treating or preventing obesity in a human patient in need of such treatment or prevention is disclosed wherein the compounds have an ion channel activity level greater than about 2 μM in the Na, K and Ca ion channels.

In another aspect of the invention, a method of treating or preventing obesity in a human patient in need of such treatment or prevention is disclosed wherein the first compound administered antagonizes the CB1 receptor at an IC50 of about 100 nM or less.

In another aspect of the invention, a method of treating or preventing obesity in a human patient in need of such treatment or prevention is disclosed wherein the second compound administered inhibits the enzyme 11β-HSD1 at an IC50 of about 100 nM or less.

In another aspect of the invention, a method of treating or preventing obesity in a human patient in need of such treatment or prevention, is disclosed wherein the first compound administered antagonizes the CB1 receptor at an IC50 of about 100 nM or less, and said first and second compounds do not substantially inhibit the CB2 receptor.

In another aspect of the invention, a method of treating or preventing obesity in a human patient in need of such treatment or prevention is disclosed wherein the second compound administered inhibits the enzyme 11β-HSD1 at an IC50 of about 100 nM or less, and said first and second compounds do not substantially inhibit the enzyme 11β-HSD2.

In another aspect of the invention, a method of treating or preventing obesity in a human patient in need of such treatment or prevention is disclosed wherein the first compound antagonizes the CB1 receptor at an IC50 of about 100 nM or less, and antagonizes the CB2 receptor at an IC50 of at least about 300 nM.

In another aspect of the invention, a method of treating or preventing obesity in a human patient in need of such treatment or prevention is disclosed wherein the second compound administered inhibits the enzyme 11β-HSD1 at an IC50 of about 100 nM or less, and inhibits the enzyme 11β-HSD2 at an IC50 of at least about 1 μM.

More particularly, a method of treating or preventing obesity in a human patient in need of such treatment or prevention is disclosed which comprises administering to the patient a compound that antagonizes CB1 receptors and antagonizes the enzyme 11β-HSD1 in an amount effective to treat or prevent obesity, with the proviso that the compound is not SR141716A.

Even more particularly, a method of treating or preventing obesity in a human patient in need of such treatment or prevention is disclosed, with the further proviso that the compound is not AM-251.

Even more particularly, a method of treating or preventing obesity in a human patient in need of such treatment or prevention is disclosed with the further proviso that the compound is not AM-281.

It will be appreciated that the treatment or prevention of obesity further includes the treatment or prevention of eating disorders, such as bulimia nervosa and compulsive eating disorders. Thus, the present invention includes a method of treating or preventing eating disorders in a human patient, which is comprised of administering to the patient a compound that antagonizes the CB1 receptor and inhibits the enzyme 11β-HSD1 in an amount that is effective to treat or prevent the eating disorder.

The compounds used in the present invention include antagonists of CB1 receptors and as such are useful as anti-obesity and appetite suppressing agents. Moreover, the compounds used in the present invention further include inhibitors of the enzyme 11β-HSD1. Lastly, the compounds used in the present invention include those having an ion channel activity of about 2 μM or higher, preferably at least about 4 μM or higher, and even more preferably, about 6 μM or higher in the Na, K and Ca ion channels.

The terms “administration of” and or “administering a” compound should be understood to mean providing a compound having the desired combination of activities, or a combination of compounds having the desired combination of activities, in an amount that is effective to treat or prevent obesity or suppress the appetite.

The term “therapeutically effective amount” means the amount of the compound or compounds that will effectively treat or prevent obesity in a patient in need of such treatment or prevention.

The dose of a compound or compounds used as described herein will, of course, vary with the nature of the severity of the condition to be treated and with the particular compound and with its route of administration. It will also vary according to the age, weight and response of the individual patient. In general, the daily dose range lies within the range of from about 0.001 mg to about 100 mg per kg body weight of the patient, preferably 0.01 mg to about 50 mg per kg, and most preferably 0.1 to 10 mg per kg, in single or divided doses. On the other hand, it may be necessary to use dosages outside these limits in some cases.

A suitable dosage range is, e.g. from about 0.01 mg to about 1000 mg of the compound per day, preferably from about 0.1 mg to about 10 mg per day. For oral administration, the composition is preferably provided in the form of tablets containing from about 0.01 to 1000 mg, preferably about 0.01, 0.05, 0.1, 0.5, 1, 2.5, 5, 10, 15, 20, 25, 30, 40, 50, 100, 250, 500, 750 or 1000 milligrams of the active ingredient, said dosage being an effective amount for the treatment or prevention of obesity or appetite suppression. While oral administration is preferred, alternative forms of administration, such as by injection, are contemplated and within the scope of the present invention as well.

The dose may be administered in a single daily dose or the total daily dosage may be administered in divided doses of two, three or four times daily. Furthermore, based on the properties of the individual compound selected for administration, the dose may be administered less frequently, e.g., weekly, twice weekly, monthly, etc.

Any suitable route of administration may be employed for providing an effective dosage of a compound or compounds of the present invention. The preferred route of administration is oral. Examples of dosage forms include tablets, troches, dispersions, suspensions, solutions, capsules and the like. When administered via intranasal routes, transdermal routes, by rectal or vaginal suppositories, or through an intravenous solution, administration can be continuous or intermittent.

Pharmaceutical compositions as described herein comprise a compound or compounds in combination with a pharmaceutically acceptable carrier. This encompasses a product comprising the active ingredient(s), and the inert ingredient(s) (pharmaceutically acceptable excipients) that make up the carrier, as well as any product which results, directly or indirectly, from the combination, complexation or aggregation of any two or more of the ingredients, or from dissociation of one or more of the ingredients, or from other types of reactions or interactions between ingredients. Accordingly, the pharmaceutical compositions of the present invention encompass any composition made by admixing a compound with additional active ingredient(s) and pharmaceutically acceptable excipients.

The pharmaceutical compositions of the present invention comprise a compound or compounds as the active ingredient or a pharmaceutically acceptable salt thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. In particular, the term “pharmaceutically acceptable salts” refers to salts prepared from pharmaceutically acceptable non-toxic bases or acids including inorganic bases or acids and organic bases or acids.

In practical use, the compounds can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the dosage forms set out above, the compounds may also be administered by controlled release means and/or delivery devices such as those described in U.S. Pat. Nos. 3,845,770; 3,916,899; 3,536,809; 3,598,123; 3,630,200 and 4,008,719.

Pharmaceutical compositions of the present invention suitable for oral administration may be presented as discrete units. Examples include tablets, capsules and cachets, each containing a predetermined amount of the active ingredient, as a powder or granules or as a solution or a suspension in an aqueous liquid, a non-aqueous liquid, an oil-in-water emulsion or a water-in-oil liquid emulsion. Methods of making the composition include the step of combining the active ingredients with the carrier. In general, the compositions are prepared by uniformly and intimately admixing the active ingredient with liquid carriers or finely divided solid carriers or both, and then, if necessary, shaping the product into the desired dosage form. For example, tablets may be prepared by compression or molding. Compressed tablets may be prepared by compressing the active ingredient in a free-flowing form such as powder or granules with the binder, lubricant, inert diluent, surface active or dispersing agent. Molded tablets may be made by molding a mixture of the powdered compound moistened with an inert liquid diluent. For example, each tablet, capsule or cachet may contain from about 0.01 to 1,000 mg, particularly 0.01, 0.05, 0.1, 0.5, 1, 2.5, 3, 5, 6, 10, 15, 25, 50, 75, 100, 125, 150, 175, 180, 200, 225, 500, 750 and 1000 milligrams of the active ingredient.

A “fast-dissolving oral formulation” is an oral delivery form which when placed on the tongue of a patient, dissolves within about 10 seconds.

The following are representative examples of pharmaceutical dosage forms useful in the present invention.

| Tablet | mg/tablet |
|---|---|
| Compound | 25 |
| Microcrystalline Cellulose | 415 |
| Povidone | 14.0 |
| Pregelatinized Starch | 43.5 |
| Magnesium Stearate | 2.5 |
| | 500 |
| Capsule | mg/capsule |
| Compound of Formula I | 25 |
| Lactose Powder | 573.5 |
| Magnesium Stearate | 1.5 |
| | 600 |

Compounds useful in the present invention may be used in combination with other drugs. Such other drugs may be administered, by a route and in an amount commonly used therefor, contemporaneously or sequentially with the compound or compounds of the present invention. When a compound or compounds are used contemporaneously with one or more other drugs, a pharmaceutical composition containing such other drugs is preferred. Accordingly, the pharmaceutical compositions of the present invention include those that also contain one or more other conventional active ingredients.

The present invention also provides a method of treating or preventing obesity, eating disorders and related metabolic disorders, which comprises administering to a patient in need of such treatment or prevention an amount of a compound of the present invention and an amount of an anorectic agent, such that together they are effective for treating or prevention the disorder.

Suitable anoretic agents of use in combination with the compounds used in the present invention include, but are not limited to, aminorex, amphechloral, amphetamine, benzphetamine, chlorphentermine, clobenzorex, cloforex, clominorex, clortermine, cyclexedrine, dexfenfluramine, dextroamphetamine, diethylpropion, diphemethoxidine, N-ethylamphetamine, fenbutrazate, fenfluramine, fenisorex, fenproporex, fludorex, fluminorex, furfurylmethylamphetamine, levamfetamine, levophacetoperane, mazindol, mefenorex, metamfepramone, methamphetamine, norpseudoephedrine, pentorex, phendimetrazine, phenmetrazine, phentermine, phenylpropanolamine, picilorex, sibutramine and pharmaceutically acceptable salts thereof.

A particularly suitable class of anorectic agent is the halogenated amphetamine derivatives, including chlorphentermine, cloforex, clortermine, dexfenfluramine, fenfluramine, picilorex and sibutramine, and pharmaceutically acceptable salts thereof.

Particularly preferred halogenated amphetamine derivatives of use in combination with a compound of the present invention include: fenfluramine and dexfenfluramine, and pharmaceutically acceptable salts thereof.

The present invention also provides a method for the treatment or prevention of obesity, which method comprises administration to a patient in need of such treatment an amount of a compound or compounds of the present invention and an amount of a selective seratonin reuptake inhibitor (SSRI), such that together they give effective relief. Suitable SSRI's for use in combination with the present invention include: fluoxetine, fluvoxamine, paroxetine and sertraline, and pharmaceutically acceptable salts thereof.

The present invention also provides for administering an amount of a compound or compounds that antagonize CB1 and inhibit 11β-HSD1 and an amount of a growth hormone secretagogue such as those described in U.S. Pat. No. 5,536,716; melanocortin agonists such as Melanotan II or those described in WO 99/64002 and WO 00/74679; 13-3 agonists such as those described in patent publications WO94/18161, WO95/29159, WO97/46556, WO98/04526 and WO98/32753; 5HT-2 agonists; orexin antagonists; melanin concentrating hormone antagonists; galanin antagonists; CCK agonists; GLP-1 agonists; corticotropin-releasing hormone agonists; NPY-5 antagonists; Y1 antagonists, histamine receptor-3 (H3) modulators, melanin concentrating hormone-1 receptor (MCH1R) antagonists, opioid receptor antagonists, lipid absorption inhibitors such as orlistat, melanin concentrating hormone-2 receptor (MCH2R) agonists and antagonists and/or phosphodiesterase-3B (PDE3B) inhibitors, such that together they are useful to treat or prevent obesity.

The present invention may also particularly be used in combination with an opioid antagonist. Examples of suitable opioid antagonists include: naloxone and nalmefene, and pharmaceutically acceptable salts thereof.

Treatment or prevention of obesity may further include the use of antidepressants, antianxiety agents, and the like. Suitable atypical antidepressants include: bupropion, lithium, nefazodone, trazodone and viloxazine, and pharmaceutically acceptable salts thereof. Suitable classes of antianxiety agents include benzodiazepines and 5-HT_{1A} agonists or antagonists, especially 5-HT_{1A} partial agonists, and corticotropin releasing factor (CRF) antagonists.

Suitable benzodiazepines include: alprazolam, chlordiazepoxide, clonazepam, chlorazepate, diazepam, halazepam, lorazepam, oxazepam and prazepam, and pharmaceutically acceptable salts thereof.

Suitable 5-HT_{1A} receptor agonists or antagonists include, in particular, the 5-HT_{1A} receptor partial agonists buspirone, flesinoxan, gepirone and ipsapirone, and pharmaceutically acceptable salts thereof.

Representative examples of compounds that can be used in the present invention include the compounds shown in tables 1 and 2. These compounds can be synthesized in accordance with the following examples.

### EXAMPLE 1

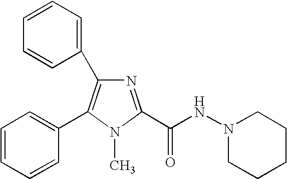

### N-(Piperidin-1-yl)-4,5-diphenyl-1-methylimidazole-2-carboxamide (Compound 1)

### Step A: 4,5-Diphenyl-1-methylimidazole-2-carboxylic Acid

To a suspension of benzyl 4,5-diphenyl-1-methylimidazole-2-carboxylate (0.43 g, 1.2 mmol) in methanol (10 mL) was added 20% palladium on carbon (50% w/w water, 110 mg) and the mixture was hydrogenated on a Parr shaker at 40 psi for 1 hr. The reaction was filtered and the filtrate was evaporated to dryness to afford the title compound as a white solid.

HPLC/MS: 279 (M+1); Rₜ=2.05 min.

### Step B: N-(Piperidin-1-yl)-4,5-diphenyl-1-methylimidazole-2-carboxamide

A mixture of 4,5-diphenyl-1-methylimidazole-2-carboxylic acid (50 mg, 0.18 mmol) from Step A, 1-aminopiperidine (0.052 mL, 0.36 mmol) (containing a small percent of piperidine as an impurity), PyBOP (NovaChem) (140 mg, 0.2 mmol) and N,N-diisopropyl-N-ethylamine (DIPEA) (0.065 mL, 0.2 mmol) in methylene chloride (2 mL) was stirred at rt for 20 hr. The reaction was diluted with water and extracted twice with methylene chloride. The organic layers were washed with brine, dried over sodium sulfate, and evaporated. The residue was purified twice by prep TLC (1 mm, silica gel) eluting with 5% methylene chloride, 35% ethyl acetate in hexanes to afford the primary product N-(piperidin-1-yl)-4,5-diphenyl-1-methylimidazole-2-carboxamide.

HPLC/MS: 361 (M+1); Rₜ=2.64 min

¹HNMR (CDCl₃): 1.4-1.7 (m, 2H), 1.92 (m, 4H), 2.96 (br s, 4H), 3.90 (s, 3H), 7.24 (m, 3H), 7.34 (m, 2H), 7.50 (m, 5H).

### EXAMPLE 2

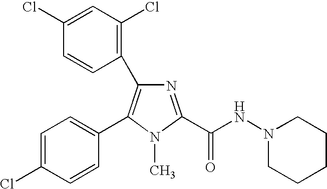

### N-(Piperidin-1-yl)-4-(2,4-dichlorophenyl)-5-(4-chlorophenyl)-1-methylimidazole-2-carboxamide (Compound 2)

Using essentially the same procedure as Step B above, (Method B), but using neat 1-aminopiperidine (3 mL), ethyl 4-(2,4-dichlorophenyl)-5-(4-chlorophenyl)-1-methylimidazole-2-carboxylate (30 mg, 0.073 mmol) was converted to the title compound after purification by Prep TLC (40% ethyl acetate in hexanes).

HPLC/MS: 463 (M+1), 465 (M+3); Rₜ=3.63 min.

### EXAMPLE 3

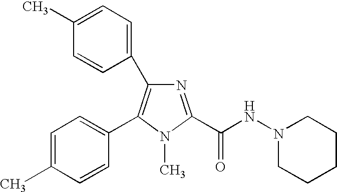

### N-(Piperidin-1-yl)-4,5-di-(4-methylphenyl)-1-methylimidazole-2-carboxamide (Compound 3)

### Step A: 4,5-di-(4-methylphenyl)-1-methylimidazole-2-carboxylic acid

To a suspension of benzyl 4,5-di-(4-methylphenyl)-1-methylimidazole-2-carboxylate (0.35 g, 0.9 mmol) in methanol (10 mL) was added 20% palladium on carbon (50% w/w water, 100 mg) and the mixture was hydrogenated at 40 psi for 1 hr. The reaction was filtered and the filtrate was evaporated to dryness to afford the title compound as a white solid.

HPLC/MS: 307 (M+1); Rₜ=2.48 min.

### Step B: N-(Piperidin-1-yl)-4,5-di-(4-methylphenyl)-1-methylimidazole-2-carboxamide

A mixture of 4,5-di-(4-methylphenyl)-1-methylimidazole-2-carboxylic acid (25 mg, 0.08 mmol) from Step A, 1-aminopiperidine (0.020 mL, 0.16 mmol) (containing a small percent of piperidine as an impurity), PyBOP (NovaChem) (65 mg, 0.1 mmol) and DIPEA (0.025 mL, 0.1 mmol) in methylene chloride (1 mL) was stirred at rt for 20 hr. The reaction was diluted with water and extracted twice with methylene chloride. The organic layers were washed with brine, dried over sodium sulfate, and evaporated. The residue was purified twice by prep TLC (1 mm, silica gel) eluting with 5% methylene chloride, 35% ethyl acetate in hexanes to afford the primary product N-(piperidin-1-yl)-4,5-di-(4-methylphenyl)-1-methylimidazole-2-carboxamide (10 mg, 32%).

HPLC/MS: 389 (M+1); Rₜ=3.04 min

### EXAMPLE 4

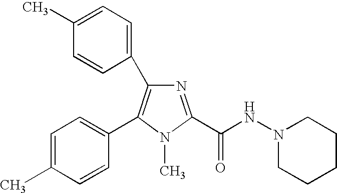

### N-Cyclohexyl-4,5-di-(4-methylphenyl)-1-methylimidazole-2-carboxamide (Compound 4)

Using cyclohexylamine (0.020 mL), 4,5-di-(4-methylphenyl)-1-methylimidazole-2-carboxylic acid (25 mg, 0.08 mmol) is converted to the title compound.

HPLC/MS: 388(M+1); Rₜ=4.19 min.

### EXAMPLE 5

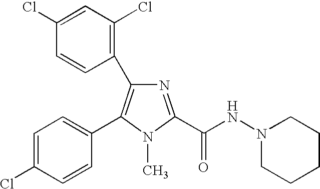

### N-(Cyclohexyl)-4-(2,4-dichlorophenyl)-5-(4-chlorophenyl)-1-methylimidazole-2-carboxamide (Compound 5)

Method A

To a solution of 4-(2,4-dichlorophenyl)-5-(4-chlorophenyl)-1-methylimidazole (50 mg, 0.15 mmol) in THF (2.5 mL) cooled to −70° C. in a dry ice/acetone bath was added 1.6N n-butyl lithium in hexanes (0.120 mL, 0.18 mmol). The reaction was stirred for 1 hr and then cyclohexyl isocyanate (0.040 mL, 0.30 mmol) was added via syringe. The reaction was allowed to warm to rt for 1 hr and was then quenched with aq. sodium bicarbonate and extracted twice with ethyl acetate. The organic layers were washed with brine, dried over sodium sulfate, and evaporated. The residue was purified by Prep TLC (2×1 mm, silica) (25% ethyl acetate in hexanes) to afford the title compound.

HPLC/MS: 462 (M+1), 464 (M+3); Rₜ=4.80 nm

¹HNMR (CDCl₃): 1.2-1.7 (4m, 6H), 1.80 (m, 2H), 2.02 (m, 21), 3.93 (m, 1H), 4.03 (s, 3H), 7.13 (dt, J=2.0 and 8.4 Hz, 2H), 7.265 (dd, J=2.1 and 8.4 Hz, 1H), 7.35 (d, J=8.4 Hz, 11), 7.37 (m, 1H), 7.39 (dt, J=2.0 and 8.4 Hz, 2H), 7.9 (v br s, 1H).

Method B

### Step A: Ethyl 4-(2,4-dichlorophenyl)-5-(4-chlorophenyl)-1-methylimidazole-2-carboxylate

To a solution of 4-(2,4-dichlorophenyl)-5-(4-chlorophenyl)-1-methylimidazole (72 mg, 0.21 mmol) in THF (3 mL) cooled to −70° C. in a dry ice/acetone bath was added 1.6N n-butyl lithium in hexanes (0.160 mL, 0.26 mmol). The reaction was stirred for 1 hr and then ethyl chloroformate (0.045 mL, 42 mmol) was added via syringe. The reaction was allowed to warm to rt for 1 hr and was then quenched with aq. sodium bicarbonate and extracted twice with ethyl acetate. The organic layers were washed with brine, dried over sodium sulfate, and evaporated. The residue was purified by Prep TLC (2×1 mm, silica) (25% ethyl acetate in hexanes) to afford the title compound.

HPLC/MS: 409 (M+1), 411 (M+3); Rₜ=3.92 min.

### Step B: N-(Cyclohexyl)-4-(2,4-dichlorophenyl)-5-(4-chlorophenyl)-1-methylimidazole-2-carboxamide

A mixture of ethyl 4-(2,4-dichlorophenyl)-5-(4-chlorophenyl)-1-methylimidazole-2-carboxylate (20 mg, 0.05 mmol) from Step A in neat cyclohexylamine (2 mL) was heated at 90° C. for 72 hr. Most of the amine was evaporated under a stream of nitrogen and the residue was purified by Prep TLC (1 mm, silica) (25% ethyl acetate in hexanes to afford the title compound.

HPLC/MS: 462 (M+1), 464 (M+3); Rₜ=4.80 min.

### EXAMPLE 6

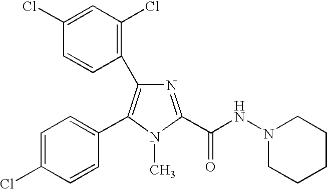

### N-(Phenyl)-4-(2,4-dichlorophenyl)-5-(4-chlorophenyl)-1-methylimidazole-2-carboxamide (Compound 6)

Using essentially the same procedure as above, Method A, but using phenyl isocyanate (0.023 mL, 0.21 mmol), 4-(2,4-dichlorophenyl)-5-(4-chlorophenyl)-1-methylimidazole (35 mg, 0.10 mmol) was converted to the title compound after purification by Prep TLC (25% ethyl acetate in hexanes).

HPLC/MS: 456 (M+1), 458 (+3); Rₜ=4.75 min.

### EXAMPLE 7

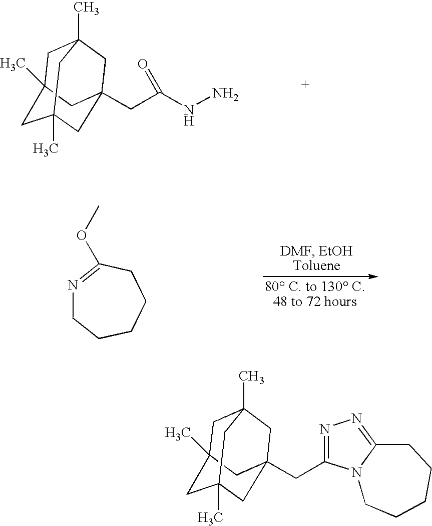

### 3-[(3,5,7-trimethyl-1-adamantyl)methyl]-6,7,8,9-tetrahydro-5H-[1,2,4]triazolo[4,3-a]azepine Trifluoroacetate Salt (7)

The referenced compound was synthesized as part of a 2-D, single, pure compound library using a Myriad Core System. All reaction vessels were dried under a stream of nitrogen at 120° C. for 12 hours prior to use. All solvents were dried over sieves for at least 12 hours prior to use. All subunits (imino ethers and acyl hydrazides) were dissolved in appropriate solvents immediately prior to use. The following table details the amounts of the subunits and solvents used in the preparation of the library:

| Substance | Amount | | MW | Concentration | Mmoles | Equivalents |
|---|---|---|---|---|---|---|
| Anhydrous | 2.8 | mL | N/A | N/A | N/A | N/A |
| Ethanol | | | | | | |
| X-axis | 0.48 | mL | N/A | 0.25 M in | 0.12 | 1.2 |
| Iminoether | | | | Anhydrous | | |
| | | | | Ethanol | | |
| Y-axis | 0.71 | mL | N/A | 0.14 M in 2.5:1 | 0.10 | 1.0 |
| Hydrazide | | | | DMF:EtOH | | |
| Toluene | 3 to 4 | mL | N/A | N/A | N/A | N/A |

To 10 mL fritted Myriad reaction vessels under nitrogen was added 2.8 ml of anhydrous ethanol. To each of the reaction vessels was then added an ethanolic solution of the X-component imino ethers (0.48 ml, 0.12 mmoles, 0.25 M in ethanol). Next, was added the appropriate Y-component hydrazide (0.71 mL, 0.1 mmoles, 0.14 M in 2.5:1 DMF: Ethanol). The reactions were aged for 1 hour at room temperature followed by 48 hours at 80° C., after which they were cooled to room temperature. Throughout the incubation, the reactions were gas agitated (1 second pulse of nitrogen every 1 hour.) Once cooled to room temperature, the crude reaction mixtures were analyzed by LC-MS (Method 1). Analysis by LC-MS indicated that the reactions were complete.

The crude reaction was purified by preparative HPLC using mass based detection (Method 2). The collected fractions were analyzed for purity by LC-MS (Method 3); fractions found to be greater than 90% pure were pooled into tared 40 mL EPA vials and lyophilized.

HPLC Purification Conditions:

| Analytical LC Method 1: | |
|---|---|
| Column: | MetaChem Polaris C-18A, 30 mm × 4.6 mm, |
| | 5.0 um |
| Eluent A: | 0.1% TFA in Water |
| Eluent B: | 0.1% TFA in Acetonitrile |
| Gradient: | 5% B to 95% B in 3.3 minutes, ramp back to |
| | 5% B in 0.3 min |
| Flow: | 2.5 mL/min. |
| Column Temperature: | 50° C. |
| Injection amount: | 5 μl of undiluted crude reaction mixture. |
| Detection: | UV at 220 and 254 nm. |
| | MS: API-ES ionization mode, mass scan range |
| | (100-600) |
| | ELSD: Light Scattering Detector |
| Preparative LC | |
| Method 2: | |
| Column: | MetaChem Polaris C-18A, 100 mm × 21.2 |
| | mm, 10 um |
| Eluent A: | 0.1% TFA in Water |
| Eluent B: | 0.1% TFA in Acetonitrile |
| Pre-inject Equilibration: | 1.0 min |
| Post-Inject Hold: | 1.0 min |
| Gradient: | 10% B to 100% B in 6.0 minutes, hold at 100% |
| | B for an additional 2.0 minutes, ramp back |
| | from 100% B o 10% B in 1.5 minutes. |
| Flow: | 20 mL/min. |
| Column Temperature: | ambient |
| Injection amount: | 1.5 ml of undiluted crude reaction mixture. |
| Detection: | MS: API-ES ionization mode, mass scan range |
| | (100-600), fraction collection triggered by |
| | detection of M + 1 |
| Analytical LC Method 3: | |
| Column: | MetaChem Polaris C-18A, 30 mm × 2.0 mm, |
| | 3.0 um |
| Eluent A: | 0.1% TFA in Water |
| Eluent B: | 0.1% TFA in Acetonitrile |
| Gradient: | 5% B to 95% B in 2.0 minutes, ramp back to |
| | 5% B in 0.1 min |
| Flow: | 1.75 mL/min. |
| Column Temperature: | 60° C. |
| Injection amount: | 5 μl of undiluted fraction. |
| Detection: | UV at 220 and 254 nm. |
| | MS: API-ES ionization mode, mass scan range |
| | (100-600) |

### ELSD: Light Scattering Detector

Lyophilization Parameters

Initial Freeze Setpoint: 1 hour at −70° C.
Drying Phase Condenser Setpoint: −50° C.

Drying Phase Table:

| Shelf | | |
|---|---|---|
| Temperature | | Vacuum Setpoint |
| (° C.) | Duration (minutes) | (mTorr) |
| −60 | 240 | 25 |
| −40 | 240 | 25 |
| 5 | 480 | 25 |
| 20 | 1000 | 25 |

Retention time: 2.048 min.
ESI (m/z): 327.0

### EXAMPLE 8

### 3-(1-adamantyl)-4-ethyl-5-(ethylthio)-4H-1,2,4-triazole

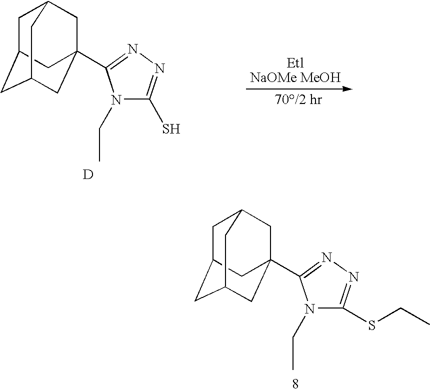

5-(1-Adamantyl)-4-ethyl-4H-1,2,4-triazole-3-thiol (D, Arzneim.-Forsch. 1991,41,1260-1264) (40 mg, 0.15 mmoles) and 0.5 M methanolic NaOMe (0.3 ml, 0.15 mmoles) in methanol (1 ml) was heated under reflux for 10 min. Ethyl iodide (12 μl, 0.15 mmoles) was added, and the mixture was heated under reflux for 2 hr. The methanol was removed in vacuo, and the residue was partitioned between CH₂Cl₂ and water. The organic layer was dried (MgSO₄) and evaporated in vacuo. The residue was purified by chromatography on silica gel with 10% MeOH in CH₂Cl₂ to give 3-(1-adamantyl)-4-ethyl-5-(ethylthio)₄H-1,2,4-triazole (8), MS: 278 (M+1).

MS ESI (m/z) 292.

### EXAMPLE 9

### 3-(1-adamantyl)-5-(3,4,5-trimethoxyphenyl)-4-methyl-4H-1,2,4-triazole

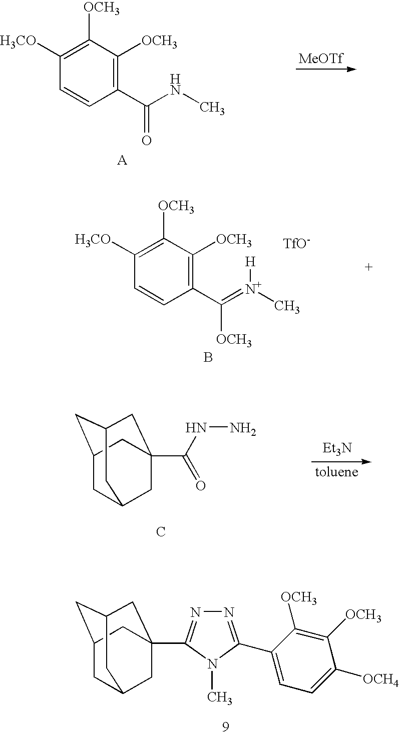

A mixture of compound (A) (16.6 mmoles) and a molar excess of methyl trifluoromethanesulfonate were reacted in a nitrogen atmosphere until the the imino ether triflate salt (B) was obtained, as confirmed by NMR. Excess methyl trifluoromethane sulfonate was removed in vacuo. Toluene (26 ml), triethylamine (3.86 ml, 27.7 mmoles) and adamantane-1-carbohydrazide (C) (2.15 g, 11.1 mmoles) were added, and the mixture was stirred at 65° C. for 5 hrs. The mixture was heated at 110° for 3 hr. The cooled reaction was diluted with ethyl acetate (75 ml), washed with water (75 ml) and saturated brine (30 ml), and dried (MgSO₄). The ethyl acetate was evaporated in vacuo. Elution with 7% methanol in chloroform and evaporation in vacuo gave compound 9. Recrystallization from isopropyl ether affords pure 9. MS: ESI (m/z) 384.

N-methyl amide starting material that is not available commercially is prepared by EDC/DMAP mediated reaction between the appropriate methyl ester or the acid chloride reacted at room temperature with 40% aqueous methylamine.

### EXAMPLE 10

### 3-adamantanyl-4,5,6,7,8,9,10,11,12,3a-decahydro-1,2,4-triazolo[4,3-a][11]annulene

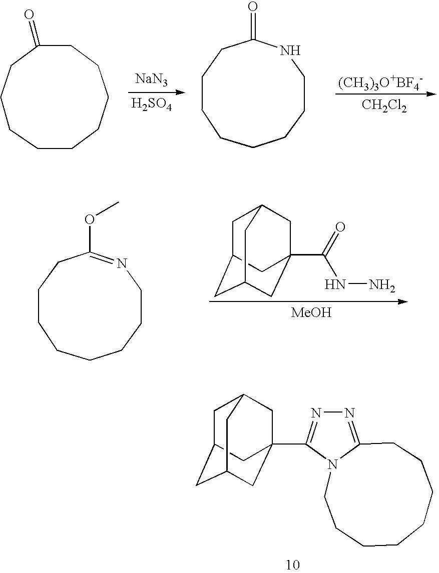

Cyclodecanone (1.0 g) in 10 mL concentrated sulfuric acid was cooled to 0° C. and 0.54 g of sodium azide was added. The reaction continued to stir at 0° C. for 1 hour and warmed to room temperature where it was stirred for two hours. The solution was diluted with cold water and treated with cold 10% NaOH solution until pH=9. Extraction with ether (2×), drying over magnesium sulfate and evaporation of solvent provided 1.23 g of 2-azacycloundecanone.

2-azacycloundecanone (0.87 g) was dissolved in 20 mL methylene chloride and stirred at room temperature under nitrogen. 1.5 g trimethyloxonium tetrafluoroborate was added and the reaction stirred overnight. The mixture was added to saturated aqueous sodium bicarbonate and extracted with methylene chloride (2×). The combined organic layers were washed with brine, dried over magnesium sulfate, and the solvent evaporated to provide crude 2-methoxyazacyclododec-1-ene.

Adamantanecarbohydrazide (45 mg) was added to a small dry flask and dissolved in 3 mL dry methanol. 63.7 mg of 2-methoxyazacyclododec-1-ene was added and the mixture was refluxed at 70° C. overnight. The methanol was removed by evaporation and 3 mL toluene added. This mixture was refluxed 24 hours at 122° C. The toluene was evaporated and the resulting solid was purified by preparative HPLC. (100% gradient/12 min) to provide 10 as the trifluoroacetate salt.

Biological activity can be demonstrated in accordance with the following assays:

Cannabinoid Receptor-1 (CB1) Binding Assay.

Binding affinity determination is based on recombinant human CB1 receptor expressed in Chinese Hamster Ovary (CHO) cells (Felder et al, Mol. Pharmacol. 48: 443450, 1995). Total assay volume is 250 μl (240 μl CB1 receptor membrane solution plus 5 μl test compound solution plus 5 μl [3H]CP-55940 solution). Final concentration of [3H]CP-55940 is 0.6 nM. Binding buffer contains 50 mM Tris-HCl, pH7.4, 2.5 mM EDTA, 5 mM MgCl₂, 0.5 mg/ml fatty acid free bovine serum albumin and protease inhibitors (Cat#P8340, from Sigma). To initiate the binding reaction, 5 μl of radioligand solution is added, the mixture is incubated with gentle shaking on a shaker for 1.5 hours at 30° C. The binding is terminated by using 96-well harvester and filtering through GF/C filter presoaked in 0.05% polyethylenimine. The bound radiolabel is quantitated using scintillation counter. Apparent binding affinities for various compounds are calculated from IC50 values (DeBlasi et al., Trends Pharmacol Sci 10: 227-229, 1989).

CB2 Receptor Binding Assay

The binding assay for CB2 receptor is conducted similarly, with recombinant human CB2 receptor expressed in CHO cells.

A suitable assay for demonstrating 11β-HSD1 and 11β-HSD2 inhibition is set forth below.

11β-HSD1 Inhibition Constants

In vitro enzymatic activity is assessed for test compounds via a Scintillation Proximity Assay (SPA). Tritiated-cortisone substrate, NADPH cofactor and titrated compound are incubated with 11β-HSD1 enzyme at 37° C. to allow conversion to cortisol to progress. Following this incubation, protein A coated SPA beads, pre-blended with anti-cortisol monoclonal antibody and a non-specific 11β-HSD inhibitor, are added to each well. The mixture is shaken at 15° C. and then read on a liquid scintillation counter suitable for 96 well plates.

Percent inhibition is calculated relative to a non-inhibited control well, and IC₅₀ curves are generated.

This assay is similarly applied to 11β-HSD2. Tritiated cortisol and NAD are used as the substrate and cofactor, respectively. 40 μL of substrate (25 nM ³H-Cortisone+1.25 mM NADPH in 50 mM HEPES Buffer, pH 7.4) are added to designated wells on a 96 well plate. Solid compound is dissolved in DMSO at 10 mM followed by a subsequent 50 fold dilution in DMSO. The diluted material is then titrated 4 fold, seven times. 1 μL of each titrated compound is then added in duplicate to the substrate. To start the reaction, 10 μL of 11β-HSD1 microsome from CHO transfectants is added to each well at the appropriate concentration to yield approximately 10% conversion of the starting material. For ultimate calculation of percent inhibition, a series of wells is added that represent the assay minimum and maximum: one set that contains substrate without compound or enzyme (background), and another set that contains substrate and enzyme without any compound (maximum signal). The plates are spun briefly at a low speed in a centrifuge to pool the reagents, sealed with an adhesive strip, mixed gently, and incubated at 37° C. for 2 hours. After incubation, 45 μL of SPA beads, pre-suspended with anti-cortisol monoclonal antibody and non-specific 11β-HSD inhibitor, are added to each well. The plates are resealed and shaken gently for greater than 1.5 hours at 15° C. Data is collected on a plate based liquid scintillation counter such as a Topcount. To control for inhibition of anti-cortisol antibody/cortisol binding, substrate spiked with 1.25 nM [3]H cortisol is added to designated single wells. 1 μL of 200 μM compound is added to each of these wells, along with 10 μL of buffer instead of enzyme. Any calculated inhibition is due to compound interfering with the cortisol binding to the antibody on the SPA beads.

Measurement of In Vivo Inhibition

In general terms, a test compound is dosed orally to a mammal and a prescribed time interval is allowed to elapse, usually between 1 and 24 hours. Tritiated cortisone is injected intravenously, followed several minutes later by blood collection. Steroids are extracted from the separated serum and analyzed by HPLC. The relative levels of ³H-cortisone and its reduction product, ³H-cortisol, are determined for compound and vehicle-dosed control groups. The absolute conversion, as well as percentage of inhibtion, are calculated from these values.

Compounds are dissolved in vehicle (5% hydroxypropyl-beta-cyclodextrin v/v H₂O, or equivalent) at the desired concentration to allow dosing at 10 milligrams per kilogram. Following an overnight fasting, the solutions are dosed to ICR mice (obtained from Charles River) by oral gavage, 0.5 mL per dose per animal, with three animals per test group.

After the desired time, either 1 or 4 hours, 0.2 mL of 3 μM ³H-cortisone in dPBS is injected by tail vein. The animal is caged for two minutes followed by euthanasia in a CO₂ chamber. Upon expiration, the mouse is removed and blood collected by cardiac puncture. The blood is set aside in a serum separation tube for no less than 30 minutes at room temperature to allow for adequate coagulation. After the incubation period, blood is separated into serum by centrifugation at 3000×g, 4° C., for 10 minutes.

To analyze the steroids in the serum, they are first extracted with organic solvent. A 0.2 mL volume of serum ias transferred to a clean microcentrifuge tube. To this, a 1.0 mL volume of ethyl acetate is added, followed by vigorous vortexing for 1 minute. The aqueous serum proteins are pelleted on a microcentrifuge, and 0.85 mL of the upper organic phase ias transferred to a fresh microcentrifuge tube and dried. The dried sample is resuspended in 0.250 mL of DMSO containing a high concentration of cortisone and cortisol for analysis by HPLC.

A 0.200 mL sample is injected onto a Metachem Inertsil C-18 chromatography column equilibrated in 30% methanol. A slow linear gradient to 50% methanol separated the target steroids; simultaneous monitoring by UV at 254 nM of the cold standards in the resuspension solution acts as an internal standard. The tritium signal is collected by a radiochromatography detector that uploads data to software for analysis. The percent conversion of ³H-cortisone to ³H-cortisol is calculated as the ratio of AUC for cortisol over the combined AUC for cortisone and cortisol.

The utility of the compounds described herein for treating or preventing obesity may be demonstrated in animal disease models that have been reported in the literature. The following are representative. a) suppression of food intake and resultant weight loss in rats (Life Sciences 1998, 63, 113-117); b) reduction of sweet food intake in marmosets (Behavioural Pharm. 1998, 9, 179-181); c) reduction of sucrose and ethanol intake in mice (Psychopharm. 1997, 132, 104-106).

Ion Channel Activity

Ion channel assays have also been reported in the literature. The following are representative.

Calcium channel binding can be demonstrated using [3H]diltiazem in accordance with Schoemaker and Langer (1985) Eur. J. Pharmacology 111: 273-277.

Sodium channel site 2 affinity binding can be demonstrated using [3H]batrachotoxin in accordance with Catterall et al (1981) J Biol Chem 269:7124-7130 or [3H]WIN 17317-3 in accordance with Wanner et al. (1999) Biochemistry 38: 11137-11146.

Potassium channel (Ikr) binding affinity can be demonstrated using [3H]dofetilide in accordance with Finlayso et al., (2001) Eur J. Pharmacol. 412: 203-212 or in accordance with PCT WO 02/05860 published on Jan. 24, 2002.

Representative examples of data for compounds that are useful in the present invention are set forth below.

TABLE 1
CB Receptor Data

| Cpd | | hCB1 | hCB2 |
|---|---|---|---|
| no. | Structure | IC50 | IC50 |
| 2 | 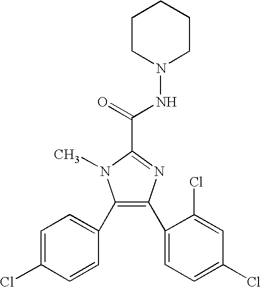 | + | +++ |
| 3 | 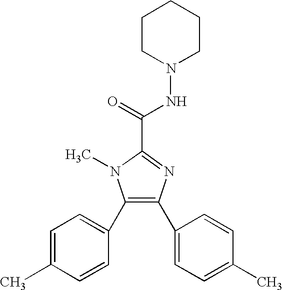 | +++ | ++++ |
| 4 | 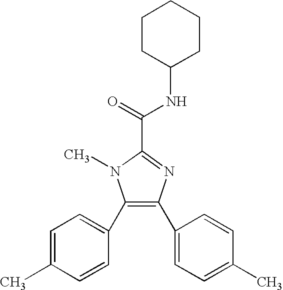 | +++ | ++++ |
| 5 | 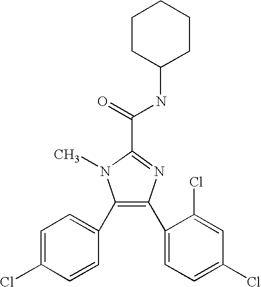 | + | +++ |
| 6 | 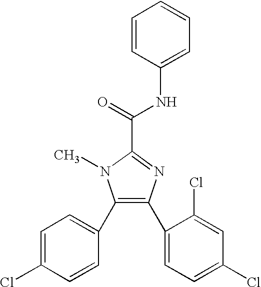 | ++ | +++ |

TABLE 2

| | IC50 | IC50 |
|---|---|---|
| Compound | hHSD1 | hHSD2 |
| Compound 7 | | |
| 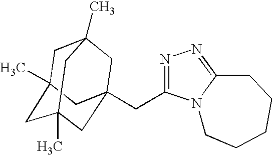 | ++ | ++++ |
| Compound 8 | | |
| 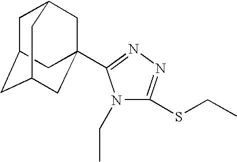 | ++ | ++++ |
| Compound 9 | | |
| 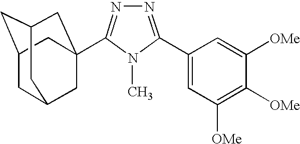 | + | + |
| Compound 10 | | |
| 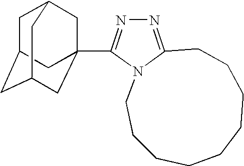 | +++ | ++++ |

+ represents ≦ 10 nM; ++ represents > 10 nM to ≦ 100 nM; +++ represents > 100 nM to ≦ 1000 nM, and ++++ represents > 1000 nM.

TABLE 3
Ion Channel Data

| | | Ion Channel |
|---|---|---|
| Cpd no. | Structure | Values |
| 1 | 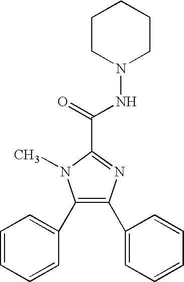 | ND |
| 2 | 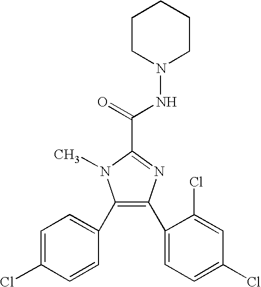 | K = 1300 Ca = 1200 Na = 6000 |
| 3 | 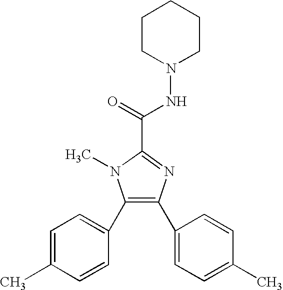 | ND |
| 4 | 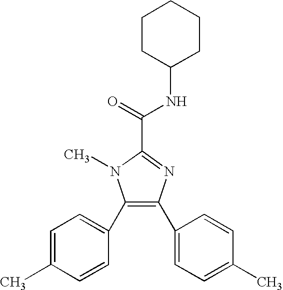 | ND |
| 5 | 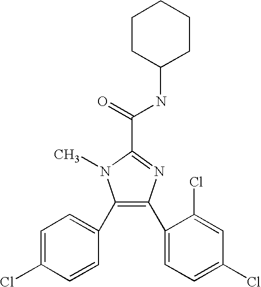 | K = 5600 Ca = 4600 Na = 4000 |

For reference purposes, SR141716A, AM-251 and AM-281 have the following structures.

| Reference Compounds | | |
|---|---|---|
| SR141716A | AM-251 | AM-281 |
| 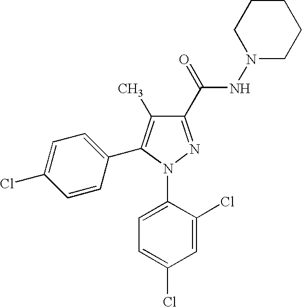 | 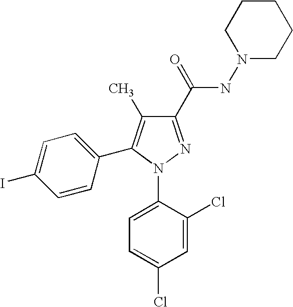 | 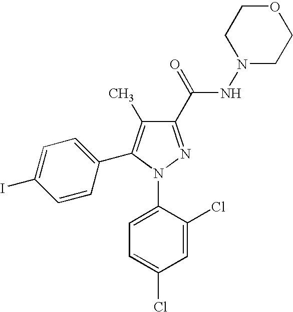 |

All publications, patents, pending patent applications and published applications, including published PCT applications, that are cited in this application are hereby incorporated by reference in their entirety.

1. A method of treating obesity in a human patient in need of such treatment, comprising administering to said patient a compound that antagonizes the CB1 receptor and inhibits the enzyme 11β-HSD1 in an amount that is effective to treat obesity, said compound having an ion channel activity level greater than about 2 μM.

2. A method of treating obesity in a human patient in need of such treatment, in accordance with claim 1, wherein the ion channel is the Na, K or Ca ion channel.

3. A method of treating obesity in a human patient in need of such treatment, in accordance with claim 2 wherein the compound has an ion channel activity level greater than about 2 μM in the Na, K and Ca ion channels.

4. A method of treating obesity in a human patient in need of such treatment in accordance with claim 3 wherein the compound is a selective antagonist of the CB1 receptor and a selective inhibitor of the enzyme 11β-HSD1.

5. A method of treating obesity in a human patient in accordance with claim 4 wherein the compound that antagonizes the CB1 receptor is at least about 10 fold selective for the CB1 receptor over the CB2 receptor.

6. A method of treating obesity in a human patient in accordance with claim 5 wherein the compound that antagonizes the CB1 receptor is from about 10 fold selective to about 1000 fold selective for the CB1 receptor over the CB2 receptor.

7. A method of treating obesity in a human patient in accordance with claim 4 wherein the compound that selectively inhibits the enzyme 11β-HSD I is at least about 10 fold selective for 11β-HSD1 over the enzyme 11β-HSD2.

8. A method of treating obesity in a human patient in accordance with claim 6 wherein the compound that inhibits the enzyme 11β-HSD I is from about 10 fold selective to about 1000 fold selective for the enzyme 11β-HSD I over the enzyme 11β-HSD2.

9. A method of treating obesity in a human patient in need of such treatment or prevention, in accordance with claim 4 wherein the compound administered antagonizes the CB1 receptor at an IC50 of about 100 nM or less.

10. A method of treating obesity in a human patient in need of such treatment, in accordance with claim 4 wherein the compound administered selectively inhibits the enzyme 11β-HSD1 at an IC50 of about 100 nM or less.

11. A method of treating obesity in a human patient in need of such treatment, in accordance with claim 4 wherein the compound administered that antagonizes the CB1 receptor and inhibits the enzyme 11β-HSD1 does not substantially antagonize the receptor CB2.

12. A method of treating obesity in a human patient in need of such treatment, in accordance with claim 11 wherein the compound administered does not substantially inhibit the enzyme 11β-HSD2.

13. A method of treating obesity in a human patient in need of such treatment, in accordance with claim 11 wherein the compound administered has an IC50 against CB2 receptors of about 300 nM or higher.

14. A method of treating obesity in a human patient in need of such treatment in accordance with claim 12 wherein the compound administered has an IC50 against the enzyme 11β-HSD2 of at least about 1 μM.

15. A method of treating obesity in a human patient in need of such treatment, comprising administering to said patient a first compound that selectively antagonizes the CB1 receptor in combination with a second compound that selectively inhibits the enzyme 11β-HSD1, said compounds being administered in combination in an amount that is effective to treat obesity, said compounds having an ion channel activity level that is greater than about 2 μM.

16. A method of treating obesity in a human patient in need of such treatment, in accordance with claim 15, wherein said compounds have an ion channel activity level that is greater than about 2 μM.

17. A method of treating obesity in a human patient in need of such treatment, in accordance with claim 15, wherein the ion channel is the Na, K or Ca ion channel.

18. A method of treating obesity in a human patient in need of such treatment, in accordance with claim 15 wherein the compounds have an ion channel activity level greater than about 2 μM in the Na, K and Ca ion channels.

19. A method of treating obesity in a human patient in need of such treatment, in accordance with claim 15 wherein the first compound administered antagonizes the CB1 receptor at an IC50 of about 100 nM or less.

20. A method of treating obesity in a human patient in need of such treatment, in accordance with claim 15 wherein the second compound administered inhibits the enzyme 11β-HSD1 at an IC50 of about 100 nM or less.

21. A method of treating obesity in a human patient in need of such treatment, in accordance with claim 19 wherein the first compound administered antagonizes the CB1 receptor at an IC50 of about 100 nM or less, and said first and second compounds do not substantially inhibit the CB2 receptor.

22. A method of treating obesity in a human patient in need of such treatment, in accordance with claim 15 wherein the second compound administered inhibits the enzyme 11β-HSD1 at an IC50 of about 100 nM or less, and said first and second compounds do not substantially inhibit the enzyme 11β-HSD2.

23. A method of treating obesity in a human patient in need of such treatment, in accordance with claim 21 wherein the first compound antagonizes the CB1 receptor at an effective concentration of about 100 nM or less, and antagonizes the CB2 receptor at an IC50 of at least about 300 nM.

24. A method of treating obesity in a human patient in need of such treatment, in accordance with claim 23, wherein the second compound administered inhibits the enzyme 11β-HSD1 at an IC50 of about 100 nM or less, and inhibits the enzyme 11β-HSD2 at a concentration of at least about 2 μM.

25. A method of treating obesity in a human patient in need of such treatment, comprising administering a compound that antagonizes CB1 receptors and antagonizes the enzyme 11β-HSD1 in an amount effective to treat obesity, with the proviso that the compound is not SR141716A.

26. A method of treating obesity in a human patient in need of such treatment, in accordance with claim 25, with the further proviso that the compound is not AM-251.

27. A method of treating obesity in a human patient in need of such treatment, in accordance with claim 26, with the further proviso that the compound is not AM-281.

28. A method of preventing obesity in a human patient in need of such prevention, comprising administering to said patient a compound that antagonizes the CB1 receptor and inhibits the enzyme 11β-HSD1 in an amount that is effective to prevent obesity, said compound having an ion channel activity level greater than about 2 μM.
